(19) United States
(12) Patent Application Publication
(10) Pub. No.: US 2026/0026785 A1
(43) Pub. Date: Jan. 29, 2026

(54) ULTRASOUND PROBE AND ULTRASONIC DIAGNOSIS APPARATUS

(71) Applicant: CANON MEDICAL SYSTEMS CORPORATION, Otawara-shi (JP)

(72) Inventors: Makoto OGI, Nasushiobara (JP); Kengo OKADA, Nasushiobara (JP); Sakie TAKEDA, Hadano (JP)

(73) Assignee: CANON MEDICAL SYSTEMS CORPORATION, Otawara-shi (JP)

(21) Appl. No.: 19/278,930

(22) Filed: Jul. 24, 2025

(30) Foreign Application Priority Data
Jul. 26, 2024 (JP) 2024-120936
May 22, 2025 (JP) 2025-085341

(51) Int. Cl. A61B 8/00

(52) U.S. Cl. CPC A61B 8/4494; A61B 8/4444

(57) ABSTRACT

An ultrasound probe according to an embodiment includes a transducer part, a contact part, and an acoustic transmission layer. The transducer part has plural transducers that transmit and receive ultrasound. The contact part comes into contact with a subject. The acoustic transmission layer is arranged between the transducer part and the contact part and is formed of a material low in ultrasonic attenuation rate. Furthermore, the acoustic transmission layer has a thickness in a range of 2.3 mm to 4.7 mm and is formed using a material having an acoustic impedance of 1.4 MRayl or more and 1.6 MRayl or less.

### CROSS-REFERENCE TO RELATED APPLICATIONS

This application is based upon and claims the benefit of priority from Japanese Patent Application No. 2024-120936, filed on Jul. 26, 2024; and Japanese Patent Application No. 2025-085341, filed on May 22, 2025; the entire contents of all of which are incorporated herein by reference.

### FIELD

Embodiments described herein relate generally to ultrasound probes and ultrasonic diagnosis apparatuses.

### BACKGROUND

Conventionally, ultrasound probes preferably have large acoustic effective apertures for ultrasound radiated by the ultrasound probes. In addition, sector ultrasound probes preferably have small contact surfaces for contact with living bodies.

An ultrasound probe, which has an acoustic transmission layer that transmits ultrasound generated by transducers, has been disclosed. The acoustic transmission layer has, on one side thereof, a curved surface formed to protrude toward a subject, the one side being near the subject. The ultrasound probe is thereby able to have: a large acoustic effective aperture; and a small contact surface for contact with a living body.

However, butadiene rubber, a material low in acoustic attenuation rate and used in the acoustic transmission layer, has low resistance to chemicals. Therefore, there is a demand for a technique to minimize deterioration of the acoustic transmission layer.

### BRIEF DESCRIPTION OF THE DRAWINGS

FIG. 1 is a block diagram illustrating an example of an ultrasonic diagnosis apparatus according to a first embodiment;

FIG. 2 is a diagram illustrating an example of a configuration of an ultrasound probe according to the first embodiment;

FIG. 3 is a diagram illustrating an example of a configuration of a conventional ultrasound probe;

FIG. 4 is a diagram illustrating an example of a configuration of an ultrasound probe according to a first modified example;

FIG. 5 is a diagram illustrating an example of a configuration of an ultrasound probe according to a second modified example; and

FIG. 6 is a diagram illustrating an example of a configuration of an ultrasound probe according to a third modified example.

### DETAILED DESCRIPTION

An ultrasound probe according to an embodiment includes a transducer part, a contact part, and an acoustic transmission layer. The transducer part has plural transducers that transmit and receive ultrasound. The contact part comes into contact with a subject. The acoustic transmission layer is arranged between the transducer part and the contact part and is formed of a material primarily containing butadiene rubber low in ultrasonic attenuation rate. The acoustic transmission layer has a thickness in a range of 2.3 mm to 4.7 mm, and the acoustic transmission layer is formed using a material having an acoustic impedance of 1.4 MRayl or more and 1.6 MRayl or less.

Ultrasound probes and ultrasonic diagnosis apparatuses, which are related to embodiments described herein, will hereinafter be described while reference is made to the drawings. Any parts in the following embodiments and assigned with the same reference sign will be assumed to operate similarly to each other and redundant description thereof will thus be omitted as appropriate.

### First Embodiment

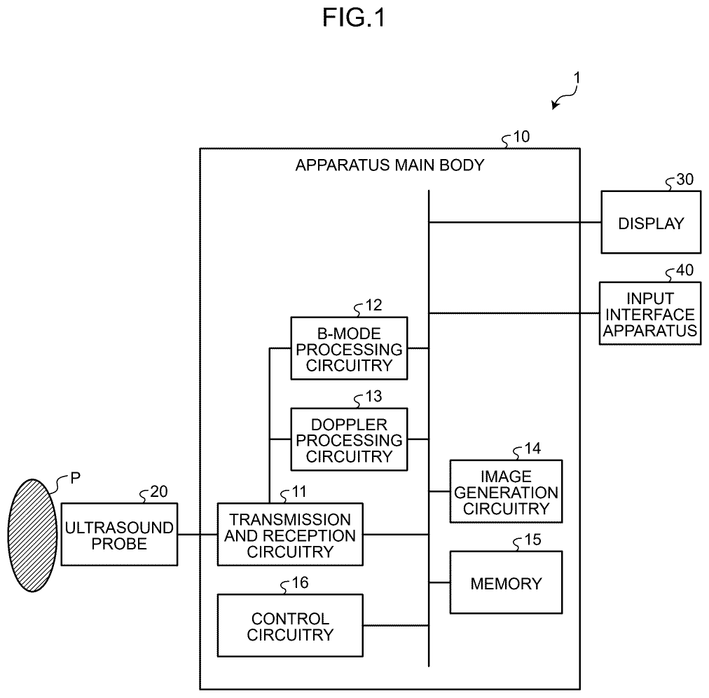

An example of a configuration of an ultrasonic diagnosis apparatus 1, to which an ultrasound probe 20 according to a first embodiment has been applied, will be described first. FIG. 1 is a block diagram illustrating an example of the ultrasonic diagnosis apparatus according to the first embodiment. The ultrasonic diagnosis apparatus 1 has an apparatus main body 10, an ultrasound probe 20, a display 30, and an input interface apparatus 40.

The ultrasound probe 20 is detachably connected to the apparatus main body 10. When ultrasound is transmitted from the ultrasound probe 20 to a subject P, the ultrasound transmitted is successively reflected by acoustic impedance discontinuities in tissue in the body of the subject P. The ultrasound reflected is received as echoes (reflected waves) by the ultrasound probe 20. For example, the ultrasound probe 20 is a two-dimensional array probe.

The display 30 displays a graphical user interface (GUI) for a user of the ultrasonic diagnosis apparatus 1 to input various setting requests using the input interface apparatus 40 and displays an ultrasound image generated in the apparatus main body 10, for example. The display 30 is implemented by a liquid crystal monitor or an organic light emitting diode (OLED) monitor, for example.

The input interface apparatus 40 is implemented by any of a trackball, switches, a dial, a touch command screen, foot switches, and a joystick, for example. The input interface apparatus 40 receives various setting requests from a user of the ultrasonic diagnosis apparatus 1 and transfers the various setting requests received, to the apparatus main body 10. For example, the input interface apparatus 40 receives various setting requests for controlling the ultrasound probe 20 and transfers the various setting requests received, to control circuitry 16.

The apparatus main body 10 is an apparatus that controls transmission and reception of ultrasound by the ultrasound probe 20 and generates an ultrasound image on the basis of an echo signal based on echoes received by the ultrasound probe 20. The apparatus main body 10 has, as illustrated in FIG. 1, transmission and reception circuitry 11, B-mode processing circuitry 12, Doppler processing circuitry 13, image generation circuitry 14, a memory 15, and control circuitry 16.

Under control by the control circuitry 16, the transmission and reception circuitry 11 implements transmission and reception of various data between the ultrasound probe 20 and the apparatus main body 10. For example, the transmission and reception circuitry 11 has an A/D converter and a received beam former. In response to the transmission and reception circuitry 11 receiving an echo signal, which is for each subarray and has been output from the ultrasound probe 20, the A/D converter converts the echo signal into digital data first. The received beam former generates echo data by subjecting digital data for each subarray to delay-and-sum processing and transmits the echo data generated, to the B-mode processing circuitry 12 and the Doppler processing circuitry 13.

The B-mode processing circuitry 12 receives echo data output from the transmission and reception circuitry 11. The B-mode processing circuitry 12 subjects the received echo data to logarithmic amplification and envelope detection processing, for example, to generate data (B-mode data) representing brightness corresponding to signal intensity. The B-mode processing circuitry 12 is implemented by, for example, a processor.

The Doppler processing circuitry 13 receives echo data output from the transmission and reception circuitry 11. The Doppler processing circuitry 13 performs frequency analysis of velocity information from the echo data received, extracts blood flow, tissue, and contrast agent echo components resulting from the Doppler effect and generates data (Doppler data) having moving object information, such as the mean velocity, variance, and power, extracted for multiple points. The Doppler processing circuitry 13 is implemented by, for example, a processor.

The image generation circuitry 14 generates an ultrasound image from data generated by the B-mode processing circuitry 12 and the Doppler processing circuitry 13. That is, the image generation circuitry 14 generates an ultrasound image on the basis of ultrasound transmitted and received by the ultrasound probe 20. The image generation circuitry 14 is an example of a generation unit. That is, the image generation circuitry 14 generates a B-mode image representing luminance corresponding to intensity of echoes from B-mode data generated by the B-mode processing circuitry 12. Furthermore, the image generation circuitry 14 generates a color Doppler image that is a mean velocity image, a variance image, a power image, or an image resulting from combination of any of these images, from Doppler data generated by the Doppler processing circuitry 13, the color Doppler image representing moving object information. The image generation circuitry 14 is implemented by, for example, a processor.

The memory 15 is implemented by, for example: a semiconductor memory element, such as a random access memory (RAM) or a flash memory; a hard disk; and/or an optical disk. For example, the memory 15 stores an ultrasound image generated by the image generation circuitry 14. The memory 15 may also store data generated by the B-mode processing circuitry 12 and the Doppler processing circuitry 13.

The control circuitry 16 controls the overall processing by the ultrasonic diagnosis apparatus 1. For example, by controlling the ultrasound probe 20 via the transmission and reception circuitry 101, the control circuitry 16 controls ultrasound scanning.

The term, “processor”, used in the above description means, for example: a central processing unit (CPU); a graphics processing unit (GPU); or a circuit, such as an application specific integrated circuit (ASIC), a programmable logic device (for example, a simple programmable logic device (SPLD), a complex programmable logic device (CPLD), or a field programmable gate array (FPGA)). A processor implements a function by reading a program stored in the memory 15 and executing the program read. Instead of being stored in the memory 15, the program may be directly incorporated in a circuit of the processor. In this case, by reading and executing the program incorporated in the circuit, the processor implements the function. Each processor in the embodiment is not necessarily configured as a single circuit, and plural independent circuits may be combined together to be configured as a single processor and to implement their functions.

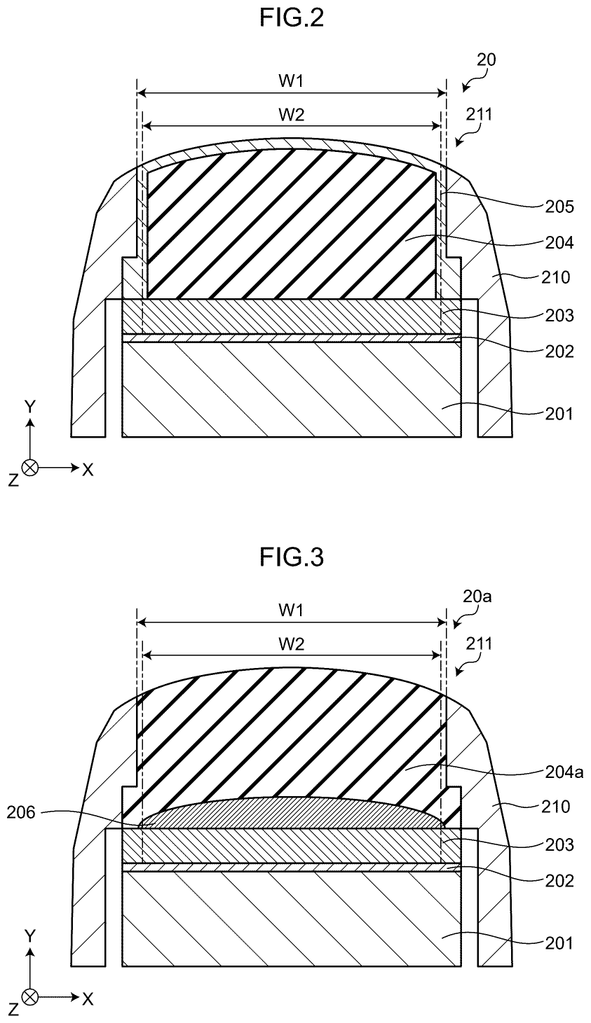

The ultrasound probe 20, which has been connected to the ultrasonic diagnosis apparatus 1, will be described next. FIG. 2 is a diagram illustrating an example of a configuration of the ultrasound probe 20 according to the first embodiment.

As illustrated in FIG. 2, the ultrasound probe 20 includes a backing unit 201, a transducer unit 202, an acoustic matching unit 203, an acoustic transmission unit 204, and a contact unit 205.

A width direction of the ultrasound probe 20 will be referred to as an X-axis direction, as illustrated in FIG. 2. A depth direction orthogonal to the X-axis direction will be referred to as a Z-axis direction. A height direction orthogonal to the X-axis direction and the Z-axis direction will be referred to as a Y-axis direction. In the ultrasound probe 20, where the contact unit 205 has been arranged will be referred to as being in front and where the backing unit 201 has been arranged will be referred to as being in back.

The backing unit 201 is arranged in back of the transducer unit 202. The backing unit 201 has a backing material. That is, the backing unit 201 absorbs ultrasound generated by the transducer unit 202. The backing unit 201 thereby minimizes return of ultrasound generated by the transducer unit 202 to the transducer unit 202, the return resulting from reflection.

The transducer unit 202 has plural transducers that transmit and receive ultrasound. The transducer unit 202 is an example of a transducer part. For example, the transducers are piezoelectric elements. For example, the transducer unit 202 has a two-dimensional array having the plural transducers arrayed two-dimensionally. More specifically, the transducer unit 202 has the plural transducers arranged along an azimuth direction. In other words, the plural transducers are arranged along the Z-axis direction. The transducer unit 202 has a width of 6.0 mm or more and 20.0 mm or less, the width being in an elevation direction orthogonal to the azimuth direction. That is, the transducer unit 202 has the width of 6.0 mm or more and 20.0 mm or less in the X-axis direction. The transducer unit 202 generates ultrasound by application of voltage to the transducers on the basis of control by the apparatus main body 10. The transducer unit 202 converts reflected waves reflected by the subject P into an electric signal. The transducer unit 202 outputs the electric signal to the apparatus main body 10.

The acoustic matching unit 203 is a layer having one or plural acoustic matching layers. The acoustic matching unit 203 is arranged in front of the transducer unit 202. In other words, the acoustic matching unit 203 is arranged between the subject P and the transducer unit 202. The acoustic matching unit 203 has one or plural acoustic matching layers having an acoustic impedance intermediate between those of the subject P and the transducer unit 202.

The acoustic impedances of the subject P and the transducer unit 202 largely differ from each other. A large difference between these acoustic impedances causes ultrasound to be reflected at a surface of the subject P. The acoustic matching unit 203 is arranged between the subject P and the transducer unit 202 and its acoustic impedance intermediate between those of the subject P and the transducer unit 202 decreases the acoustic impedance difference. The acoustic matching unit 203 thereby enables ultrasound to more easily enter the subject P.

The acoustic transmission unit 204 is a layer to transmit ultrasound generated by the transducer unit 202. More specifically, the acoustic transmission unit 204 is arranged between the transducer unit 202 and the contact unit 205 and is formed of a material low in ultrasonic attenuation rate. For example, the acoustic transmission unit 204 is formed of a material primarily containing butadiene rubber. For example, the acoustic transmission unit 204 is formed of a material having an acoustic attenuation rate of 0.003 dB/mmMHz or less.

Specifically, the material for the acoustic transmission unit 204 preferably:
(a) propagates ultrasound at a velocity of 1500 m/sec or more and 1600 m/sec or less;
(b) has an acoustic impedance of 1.4 MRayl or more and 1.6 MRayl or less; and
(c) has attenuation of about 0.03×0.10 dB/mmMHz.

The acoustic transmission unit 204 preferably has a thickness of 2.3 mm or more. That is, the acoustic transmission unit 204 preferably has a length of 2.3 mm or more in the Y-axis direction. A lateral surface unit 210 and the contact unit 205 are bonded to each other with a silicone adhesive. The contact unit 205 and the acoustic transmission unit 204 are also bonded to each other with a silicone adhesive. Silicone adhesives have waterproofness but are low in rigidity and low in adhesive strength for plastic materials and thus require comparatively large bonding areas. To obtain a sufficient bonding area, the acoustic transmission unit 204 has a thickness of 2.3 mm or more. The acoustic transmission unit 204 is thus bonded to the contact unit 205 with sufficient strength. The contact unit 205 is also bonded to the lateral surface unit 210 with sufficient strength.

The thickness of the acoustic transmission unit 204 may be 2.3 mm or more and 4.7 mm or less. Increasing the thickness, that is, the length in the Y-axis direction, of the acoustic transmission unit 204 as described above enables reduction in width of contact with the subject P.

In a case where the ultrasound probe 20 is a sector ultrasound probe, its acoustic effective aperture illustrated in FIG. 2 is preferably increased as much as possible for ultrasound to be increased in intensity. That is, the ultrasound probe 20 is desired to be increased in transducer width W2 as much as possible. Furthermore, the ultrasound probe 20 is desired to be decreased in area of a contact surface for contact with a living body for a scan of a circulatory organ from between ribs of the subject P, the contact surface being of the contact unit 205 and being a portion that comes into contact with the subject P. The contact unit 205 has a curved surface protruding forward for reduction of the area of the contact surface for contact with a living body. By being formed to be a curved surface, the contact surface of the contact unit 205 and for contact with a living body is smaller than that of a contact unit having a planar contact surface.

If the acoustic transmission unit 204 were not to be provided and the contact unit 205 had a curved surface protruding forward, outer edge of the transducer unit 202 would interfere with the contact unit 205 because the transducer unit 202 is approximately planar. Therefore, providing the acoustic transmission unit 204 enables the contact unit 205 to have a curved surface. That is, the acoustic transmission unit 204 enables reduction of the area of the contact surface for contact with a living body. The acoustic transmission unit 204 thus has a sufficient thickness for providing the curved surface on the contact unit 205. For example, the acoustic transmission unit 204 is thicker than the contact unit 205 in the Y-axis direction. In other words, the acoustic transmission unit 204 is thicker than the contact unit 205 in a direction, in which the transducer unit 202, the contact unit 205, and the acoustic transmission unit 204 have been layered over one another.

The transducer unit 202 has a width smaller than that of an aperture 211 of the lateral surface unit 210, the width being in the elevation direction orthogonal to the azimuth direction, in which the plural transducers have been arranged. That is, the width of the transducer unit 202 in the X-axis direction is smaller than the width of the aperture 211 of the lateral surface unit 210. A difference between a lateral surface unit aperture width W1, which is the width of the aperture 211 of the lateral surface unit 210 and the transducer width W2 of the transducers of the transducer unit 202 in the X-axis direction is more than 0.0 mm and 1.0 mm or less. The ultrasound probe 20 configured as described above enables minimization of attenuation of ultrasound by the lateral surface unit 210.

This small difference between the lateral surface unit aperture width W1 and the transducer width W2 in the ultrasound probe 20 enables minimization of: image quality deterioration in a portion (deep portion) away from the contact unit 205; and reduction in signal-to-noise ratio (S/N ratio).

The contact unit 205 comes into contact with the subject P. The contact unit 205 is an example of a contact part. For example, the contact unit 205 is arranged in front of the acoustic transmission unit 204. That is, the contact unit 205 is arranged between the acoustic transmission unit 204 and the subject P. The contact unit 205 has a thickness of 0.3 mm or more and 2.00 mm or less at the center of the contact unit 205. That is, the thickness of the contact unit 205 at the center of its length in the Z-axis direction and at the approximate center of its length in the X-axis direction is 0.3 mm or more and 2.0 mm or less. The amount of attenuation of ultrasound in the acoustic transmission unit 204 and the contact unit 205 is less than 1.2 dB/MHz per 1 mm. The ultrasound probe 20 thereby has an acoustic configuration lower in attenuation than typical ultrasound probes and enables high-quality images to be acquired.

For example, the contact unit 205 is formed of a material primarily containing butadiene rubber. In pure silicone rubber, ultrasound propagates at a propagation velocity of about 1000 m/sec. Furthermore, pure silicone rubber has an acoustic impedance of about 1 MRayl. Living bodies have an acoustic impedance of about 1.5 MRayl. Because pure silicone rubber is lower in acoustic impedance than living bodies, fine powder of a material high in specific gravity, such as silica, is added to pure silicone rubber. In a case where the propagation velocity, at which ultrasound propagates through silicone rubber added with a material high in specific gravity is lower than the propagation velocity, at which ultrasound propagates through living bodies, forming the silicone rubber in a convex shape allows the silicone rubber to have an effect of focusing ultrasound and the silicone rubber is thus used as an acoustic lens.

Furthermore, because silicone rubber is flexible, silicone rubber tends to transmit any impact thereon to the transducer unit 202. Therefore, the ultrasound probe 20 has the lateral surface unit 210 for protection from any impact due to the ultrasound probe 20 being dropped, for example. The lateral surface unit 210 is a member that is formed on lateral surfaces of the acoustic transmission unit 204 and that lets the contact unit 205 protrude from the aperture 211 formed at one end of the lateral surface unit 210. the lateral surface unit 210 is an example of a lateral surface part. Furthermore, the lateral surface unit 210 is a member that covers lateral surfaces of a distal end portion of the ultrasound probe 20 and that is high in rigidity. That is, the lateral surface unit 210 covers lateral surfaces of the contact unit 205. By covering the lateral surfaces of the contact unit 205, the lateral surface unit 210 fixes the contact unit 205 to a grasped portion. For example, the lateral surface unit 210 is formed of a plastic material.

The contact unit 205 and the lateral surface unit 210 are bonded to each other with a silicone adhesive. Silicone adhesives have waterproofness but are low in rigidity and low in adhesive strength for plastic materials and thus require comparatively large bonding areas. Therefore, as illustrated in FIG. 2, the contact unit 205 is provided to cover an area up to and including the lateral surfaces of the acoustic transmission unit 204. Because a comparatively large bonding area is thereby provided, the contact unit 205 is bonded to the lateral surface unit 210 with the silicone adhesive at the lateral surfaces of the acoustic transmission unit 204.

The contact surface for contact with the subject P thus has a large width at the distal end portion of the ultrasound probe 20. That is, the width of the contact surface for contact with a living body becomes large in relation to the width of the transducer unit 202, the widths being in the X-axis direction.

FIG. 3 is a diagram illustrating an example of a configuration of a conventional ultrasound probe 20a. As illustrated in FIG. 3, the conventional ultrasound probe 20a includes a backing unit 201, a transducer unit 202, an acoustic matching unit 203, an acoustic lens unit 206, and an acoustic transmission unit 204a.

That is, the conventional ultrasound probe 20a does not have the contact unit 205. Therefore, the acoustic transmission unit 204a of the conventional ultrasound probe 20a comes into contact with a subject P.

The acoustic transmission units 204 and 204a are formed of a material primarily containing butadiene rubber. However, a material primarily containing butadiene rubber has low resistance to chemicals.

After being used, the ultrasound probe 20 is subjected to treatment, such as washing, sterilization, or disinfection. In this treatment, the ultrasound probe 20 is irradiated with ultraviolet (UV) and/or sprayed with a chemical. The acoustic transmission units 204 and 204a formed of a material primarily containing butadiene rubber have low resistance to UV and chemicals.

Therefore, the ultrasound probe 20 has, as illustrated in FIG. 2, the contact unit 205 covering a front surface of the acoustic transmission unit 204. In other words, the contact unit 205 covers an exposed portion of the acoustic transmission unit 204.

The contact unit 205 is arranged to cover the front surface of the acoustic transmission unit 204, as illustrated in FIG. 2. More specifically, the contact unit 205 is arranged to cover the front surface and the lateral surfaces of the acoustic transmission unit 204. The contact unit 205 thereby protects the acoustic transmission unit 204 from UV and chemicals.

For example, the contact unit 205 is formed of a material that is the same as that of an acoustic lens. Acoustic lenses focus ultrasound beams in the X-axis direction and the Z-axis direction. Acoustic lenses thereby achieve improvement of the azimuth resolution. For example, acoustic lenses are formed of a material primarily containing butadiene rubber.

For example, the contact unit 205 is formed of a material having excellent chemical resistance. Specifically, the contact unit 205 is formed of a material primarily containing silicone rubber. Furthermore, the contact unit 205 may be formed of a material primarily containing a polyimide.

For example, the acoustic lens unit 206 of the conventional ultrasound probe 20a illustrated in FIG. 3 is formed of a material primarily containing silicone rubber. A maker thus applies a silicone-based adhesive between the acoustic lens unit 206 and the transducer unit 202, cures the silicone-based adhesive applied, and thereby bonds the acoustic lens unit 206 and the transducer unit 202 to each other. The acoustic transmission unit 204a is formed of a material primarily containing butadiene rubber. The maker thus applies a butadiene-based adhesive between the acoustic lens unit 206 and the acoustic transmission unit 204a, cures the butadiene-based adhesive applied, and thereby bonds the acoustic lens unit 206 and the acoustic transmission unit 204a to each other. That is, bonding for the conventional ultrasound probe 20a is carried out through multiple steps using different adhesives.

The acoustic transmission unit 204 of the ultrasound probe 20, on the other hand, is formed of a material primarily containing butadiene rubber. The transducer unit 202 and the acoustic transmission unit 204 are thus bonded to each other with a butadiene-based adhesive. That is, a maker applies a butadiene-based adhesive between the transducer unit 202 and the acoustic transmission unit 204, cures the butadiene-based adhesive applied, and thereby bonds the transducer unit 202 and the acoustic transmission unit 204 to each other. Furthermore, the maker applies a butadiene-based adhesive between the contact unit 205 and the acoustic transmission unit 204, cures the butadiene-based adhesive applied, and thereby bonds the contact unit 205 and the acoustic transmission unit 204 to each other. Because both of these adhesives are butadiene-based, the maker is able to carry out the bonding by curing the adhesives at once. Furthermore, because butadiene-based adhesives are smaller in acoustic attenuation rate than silicone-based adhesives, butadiene-based adhesives enable reduction in the ultrasonic attenuation rate.

As described above, the ultrasound probe 20 according to the first embodiment includes the transducer unit 202, the contact unit 205, and the acoustic transmission unit 204. The acoustic transmission unit 204 is arranged between the transducer unit 202 and the contact unit 205 and is formed of a material primarily containing butadiene low in ultrasonic attenuation rate. That is, because the acoustic transmission unit 204 is covered with the contact unit 205, the acoustic transmission unit 204 is not exposed to UV and does not come into contact with chemicals. Therefore, the ultrasound probe 20 enables minimization of deterioration of the acoustic transmission layer.

### First Modified Example

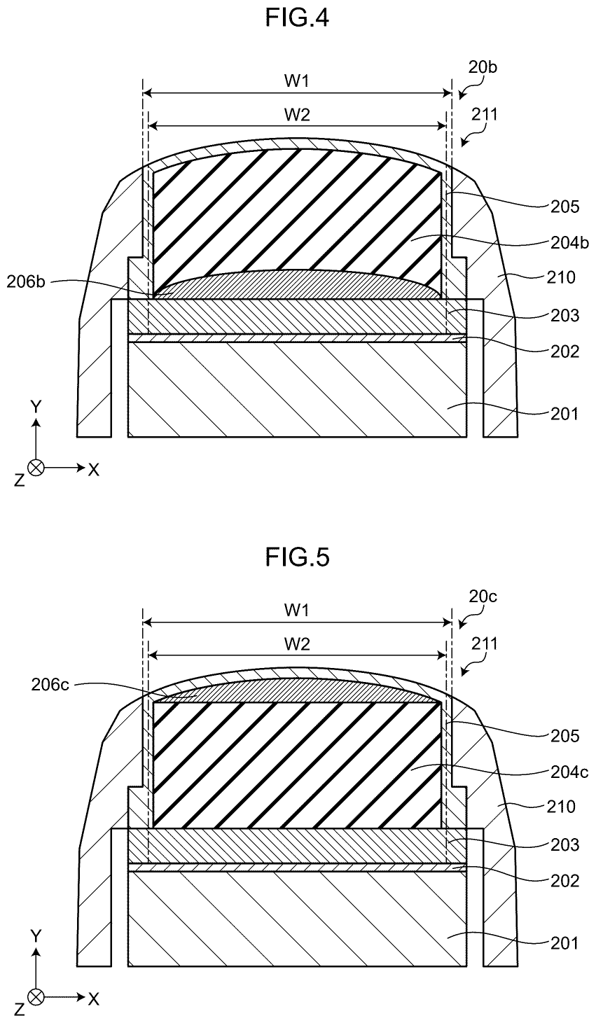

FIG. 4 is a diagram illustrating an example of a configuration of an ultrasound probe 20b according to a first modified example. As illustrated in FIG. 4, the ultrasound probe 20b may have an acoustic lens unit 206b in front of a transducer unit 202. That is, the ultrasound probe 20b may have the acoustic lens unit 206b between an acoustic transmission unit 204b and the transducer unit 202.

The acoustic lens unit 206b is arranged between the transducer unit 202 and the acoustic transmission unit 204b and focuses ultrasound transmitted by the transducer unit 202. The acoustic lens unit 206b is an example of a first acoustic lens unit. More specifically, the acoustic lens unit 206b has an acoustic lens that focuses an ultrasound beam in the X-axis direction and the Z-axis direction. The acoustic lens unit 206b thereby achieves improvement of the azimuth resolution. For example, the acoustic lens unit 206b is formed of a material primarily containing silicone rubber.

In a case where a propagation velocity, at which ultrasound propagates through the acoustic lens unit 206b, is lower than a propagation velocity, at which ultrasound propagates through a living body, a surface of the acoustic lens unit 206b has a convex shape, the surface being near the subject P and being convex. That is, the acoustic lens unit 206b has a convex shape that is convex in the Y-axis direction that is a thickness direction of the acoustic lens unit 206b. In contrast, in a case where the propagation velocity, at which ultrasound propagates through the acoustic lens unit 206b, is higher than the propagation velocity, at which ultrasound propagates through a living body, the surface of the acoustic lens unit 206b has a concave shape, the surface being near the subject P and being concave. That is, the acoustic lens unit 206b has a concave shape that is concave in the Y-axis direction that is the thickness direction of the acoustic lens unit 206b. The thickest portion of the acoustic lens unit 206b preferably has a thickness of, for example, 0.3 mm or more and 0.9 mm or less.

### Second Modified Example

FIG. 5 is a diagram illustrating an example of a configuration of an ultrasound probe 20c according to a second modified example. As illustrated in FIG. 5, the ultrasound probe 20c may have an acoustic lens unit 206c in front of an acoustic transmission unit 204c.

The acoustic lens unit 206c is arranged between the acoustic transmission unit 204c and a contact unit 205 and focuses ultrasound transmitted by a transducer unit 202. The acoustic lens unit 206c is an example of a second acoustic lens unit. More specifically, the acoustic lens unit 206c has an acoustic lens that focuses an ultrasound beam in the X-axis direction and the Z-axis direction. The acoustic lens unit 206c thereby achieves improvement of the azimuth resolution. For example, the acoustic lens unit 206c is formed of a material primarily containing silicone rubber.

In this second modified example also, a maker applies a butadiene-based adhesive between the transducer unit 202 and the acoustic transmission unit 204c, cures the butadiene-based adhesive applied, and thereby bonds the transducer unit 202 and the acoustic transmission unit 204c to each other. Furthermore, the maker applies a butadiene-based adhesive between the acoustic lens unit 206c and the acoustic transmission unit 204c, cures the butadiene-based adhesive applied, and thereby bonds the acoustic lens unit 206c and the acoustic transmission unit 204c to each other. That is, because both of these adhesives are butadiene-based, the maker is able to carry out the bonding by curing the adhesives at once.

### Third Modified Example

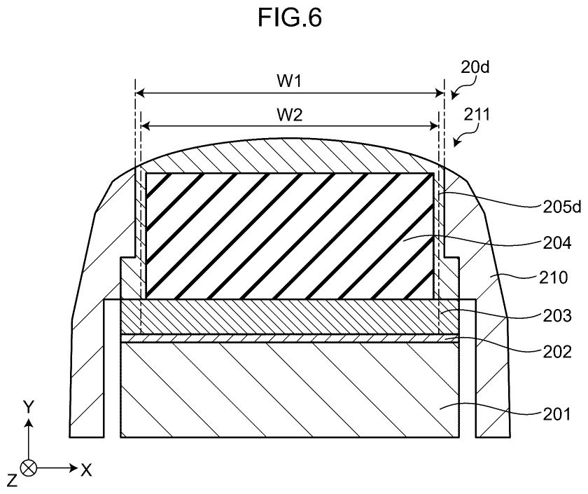

FIG. 6 is a diagram illustrating an example of a configuration of an ultrasound probe 20d according to a third modified example. As illustrated in FIG. 6, the ultrasound probe 20d may have a contact unit 205d having an ultrasonic lens.

The contact unit 205d is formed of a material primarily containing silicone rubber, similarly to the ultrasonic lens. Therefore, the contact unit 205d may function as an acoustic lens. In other words, the contact unit 205d focuses ultrasound transmitted by a transducer unit 202.

In this third modified example also, a maker applies a butadiene-based adhesive between the transducer unit 202 and an acoustic transmission unit 204, cures the butadiene-based adhesive applied, and thereby bonds the transducer unit 202 and the acoustic transmission unit 204 to each other. Furthermore, the maker applies a butadiene-based adhesive between the contact unit 205d and the acoustic transmission unit 204, cures the butadiene-based adhesive applied, and thereby bonds the contact unit 205d and the acoustic transmission unit 204 to each other. That is, because both of these adhesives are butadiene-based, the maker is able to carry out the bonding by curing the adhesives at once.

At least one of the embodiment and modified examples described above enables minimization of deterioration of an acoustic transmission layer.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

1. An ultrasound probe, comprising:
a transducer part having plural transducers that transmit and receive ultrasound;
a contact part that comes into contact with a subject; and
an acoustic transmission layer arranged between the transducer part and the contact part and formed of a material low in ultrasonic attenuation rate, wherein
the acoustic transmission layer has a thickness in a range of 2.3 mm to 4.7 mm, and
the acoustic transmission layer is formed using a material having an acoustic impedance of 1.4 MRayl or more and 1.6 MRayl or less.

2. The ultrasound probe according to claim 1, wherein an amount of attenuation of ultrasound in the acoustic transmission layer and the contact part is less than 1.2 dB/MHz per 1 mm.

3. The ultrasound probe according to claim 1, further comprising:
a lateral surface part that is formed on a lateral surface of the acoustic transmission layer and lets the contact part protrude from an aperture formed at one end of the lateral surface part, wherein
the plural transducers of the transducer part are arranged along an azimuth direction,
the transducer part have a width smaller than a width of the aperture, the widths being in an elevation direction orthogonal to the azimuth direction, and
a difference between the width of the transducers and the width of the aperture is 1.0 mm or less, the widths being in the elevation direction.

4. The ultrasound probe according to claim 1, further comprising:
a first acoustic lens between the transducer part and the acoustic transmission layer, wherein
the first acoustic lens has a thickness of 0.3 mm or more and 0.9 mm or less and has a concave surface that is concave in a thickness direction of the first acoustic lens or a convex surface that is convex in the thickness direction.

5. The ultrasound probe according to claim 1, wherein the transducer part have a width of 6.0 mm or more and 20.0 mm or less, the width being in an elevation direction orthogonal to an azimuth direction that the plural transducers have been arranged along.

6. The ultrasound probe according to claim 1, wherein
a propagation velocity of ultrasound propagated through the acoustic transmission layer ranges from 1500 m/sec to 1600 m/sec, and
the acoustic transmission layer is formed of a material having attenuation of 0.03×0.10 dB/mmMHz.

7. The ultrasound probe according to claim 1, wherein the acoustic transmission layer is formed of a material having an ultrasonic attenuation rate of 0.003 dB/mmMHz or less.

8. The ultrasound probe according to claim 1, further comprising a first acoustic lens that is arranged between the transducer part and the acoustic transmission layer and focuses ultrasound transmitted by the transducer part.

9. The ultrasound probe according to claim 1, further comprising a second acoustic lens that is arranged between the acoustic transmission layer and the contact part and focuses ultrasound transmitted by the transducer part.

10. The ultrasound probe according to claim 1, wherein the contact part focuses ultrasound transmitted by the transducer part.

11. The ultrasound probe according to claim 1, wherein the acoustic transmission layer is thicker than the contact part in a direction that the transducer part, the contact part, and the acoustic transmission layer have been layered in.

12. The ultrasound probe according to claim 1, wherein the transducer part and the acoustic transmission layer have been bonded to each other with a butadiene-based adhesive.

13. The ultrasound probe according to claim 1, wherein the contact part covers a surface of the acoustic transmission layer, the surface being near the subject.

14. The ultrasound probe according to claim 1, wherein the contact part is formed of a material primarily containing silicone rubber.

15. An ultrasonic diagnosis apparatus, comprising:
an ultrasound probe; and
an apparatus main body, wherein
the ultrasound probe comprises:
a transducer part having plural transducers that transmit and receive ultrasound;
a contact part that comes into contact with a subject; and
an acoustic transmission layer arranged between the transducer part and the contact part and formed of a material low in ultrasonic attenuation rate,
the acoustic transmission layer has a thickness in a range of 2.3 mm to 4.7 mm,
the acoustic transmission layer is formed using a material having an acoustic impedance of 1.4 MRayl or more and 1.6 MRayl or less, and
the apparatus main body includes generation circuitry that generates an ultrasound image on the basis of ultrasound transmitted and received by the transducer part.
